(19) United States
(12) Patent Application Publication
(10) Pub. No.: US 2024/0415418 A1
(43) Pub. Date: Dec. 19, 2024

(54) OPTICAL CHEMICAL SENSOR SYSTEM WITH MICRONEEDLES

(71) Applicant: Cardiac Pacemakers, Inc., St. Paul, MN (US)

(72) Inventors: Yingbo Li, Woodbury, MN (US); Michael J. Kane, St. Paul, MN (US); Kenneth Martin Stein, Minneapolis, MN (US)

(21) Appl. No.: 18/797,900

(22) Filed: Aug. 8, 2024

Related U.S. Application Data
(63) Continuation of application No. 18/736,986, filed on Jun. 7, 2024.
(60) Provisional application No. 63/472,654, filed on Jun. 13, 2023.

(51) Int. Cl. A61B 5/145; A61B 5/00; A61B 5/1459

(52) U.S. Cl. CPC A61B 5/14514; A61B 5/14539; A61B 5/14546; A61B 5/1459; A61B 5/6824; A61B 5/6833; A61B 2562/0233; A61B 2562/0271; A61B 2562/028; A61B 2562/046; A61B 2562/164; A61B 2562/168

(57) ABSTRACT

Embodiments herein relate to optical chemical sensor systems that can be wearable and include microneedle arrays. In an embodiment, a chemical sensing device can be included having an optical transmission layer. The optical transmission layer can define a plurality of microneedles. The chemical sensing device can include a plurality of sensor elements and one or more optical emitters, wherein the one or more optical emitters can be configured to emit light into the plurality of sensor elements. The chemical sensing device can also include a plurality of optical detectors, wherein the plurality of optical detectors can be configured to receive light from the plurality of sensor elements. Other embodiments are also included herein.

This application is a continuation of U.S. patent application Ser. No. 18/736,986 filed Jun. 7, 2024, which claims the benefit of U.S. Provisional Application No. 63/472,654, filed Jun. 13, 2023, the contents of all of which are herein incorporated by reference in their entirety.

### FIELD

Embodiments herein relate to chemical sensors. More specifically, embodiments herein relate to optical chemical sensor systems that can be wearable and include microneedle arrays.

### BACKGROUND

In the context of diagnosis and monitoring of patients, clinicians frequently evaluate many different pieces of data about their patients including physical observations, descriptions of symptoms, test results, and the like. One aspect that testing can reveal is the physiological concentration of chemical analytes such as electrolytes for the patient. Chemical analyte concentrations can be important to know because of their effect on various organs and bodily functions. In many cases, chemical analyte concentrations are assessed by drawing a fluid sample (or other sample) from the patient followed by an in vitro assay.

### SUMMARY

Embodiments herein relate to optical chemical sensor systems that can be wearable and include microneedle arrays. In a first aspect, a chemical sensing device can be included having a plurality of microneedles and a sensor element. The sensor element can include a first low-index gel layer, a second low-index gel layer, and a chromoionophore sensing film. The chromoionophore sensing film can be disposed between the first low-index gel layer and the second low-index gel layer. The chemical sensing device can also include a delay layer, wherein the delay layer can be disposed between the sensor element and the plurality of microneedles. The chemical sensing device can also include an optical emitter, wherein the optical emitter can be configured to emit light into the sensor element. The chemical sensing device can also include an optical detector, wherein the optical detector can be configured to receive light from the sensor element.

In a second aspect, in addition to one or more of the preceding or following aspects, or in the alternative to some aspects, the sensor element can be configured to detect at least one analyte. The at least one analyte can include at least one selected from the group consisting of potassium ion, sodium ion, hydrogen ion (pH), and creatinine.

In a third aspect, in addition to one or more of the preceding or following aspects, or in the alternative to some aspects, the chemical sensing device can further include at least one reflector and at least one filter guide. The at least one filter guide and the at least one reflector can be configured to be disposed optically between at least one of the optical emitter and the optical detector and the sensor element.

In a fourth aspect, in addition to one or more of the preceding or following aspects, or in the alternative to some aspects, the chemical sensing device can further include a pH sensor.

In a fifth aspect, in addition to one or more of the preceding or following aspects, or in the alternative to some aspects, the pH sensor can be integrated with or separate from the sensor element.

In a sixth aspect, in addition to one or more of the preceding or following aspects, or in the alternative to some aspects, the chemical sensing device can further include a temperature sensor.

In a seventh aspect, in addition to one or more of the preceding or following aspects, or in the alternative to some aspects, the temperature sensor can be integrated with or separate from the sensor element.

In an eighth aspect, in addition to one or more of the preceding or following aspects, or in the alternative to some aspects, the chemical sensing device can further include a liquid reservoir, wherein the liquid reservoir can be in fluid communication with at least one of the sensor element and the delay layer.

In a ninth aspect, in addition to one or more of the preceding or following aspects, or in the alternative to some aspects, the liquid reservoir can be in selective fluid communication with at least one of the sensor element and the delay layer.

In a tenth aspect, in addition to one or more of the preceding or following aspects, or in the alternative to some aspects, the chemical sensing device can further include a pH modifying composition, wherein the pH modifying composition can be disposed within the liquid reservoir.

In an eleventh aspect, in addition to one or more of the preceding or following aspects, or in the alternative to some aspects, the pH modifying composition can include a carboxylic acid.

In a twelfth aspect, in addition to one or more of the preceding or following aspects, or in the alternative to some aspects, the carboxylic acid can include citric acid.

In a thirteenth aspect, in addition to one or more of the preceding or following aspects, or in the alternative to some aspects, the pH modifying composition can be effective to lower the pH of the sensor element to a pH of 3 to 5.

In a fourteenth aspect, in addition to one or more of the preceding or following aspects, or in the alternative to some aspects, the chemical sensing device can further include a flow control element, wherein the flow control element can be in fluid communication with the liquid reservoir and can be configured to control a flow of the pH modifying composition from the liquid reservoir to the delay layer and/or the sensor element.

In a fifteenth aspect, in addition to one or more of the preceding or following aspects, or in the alternative to some aspects, the flow control element can include an actuatable valve.

In a sixteenth aspect, in addition to one or more of the preceding or following aspects, or in the alternative to some aspects, the flow control element can include a pump.

In a seventeenth aspect, in addition to one or more of the preceding or following aspects, or in the alternative to some aspects, the flow control element can include a mechanism to change a pressure within the liquid reservoir.

In an eighteenth aspect, in addition to one or more of the preceding or following aspects, or in the alternative to some aspects, the liquid reservoir can include discrete separately controllable liquid portions.

In a nineteenth aspect, in addition to one or more of the preceding or following aspects, or in the alternative to some aspects, the chemical sensing device can further include an adhesive patch, wherein the adhesive patch can be configured to hold the chemical sensing device onto the skin of a patient.

In a twentieth aspect, in addition to one or more of the preceding or following aspects, or in the alternative to some aspects, the chemical sensing device can further include a control circuit, wherein the control circuit can be configured to control the optical emitter and receive signals from the optical detector.

In a twenty-first aspect, in addition to one or more of the preceding or following aspects, or in the alternative to some aspects, the delay layer can be effective to delay migration of a solute from the plurality of microneedles to the sensor element.

In a twenty-second aspect, in addition to one or more of the preceding or following aspects, or in the alternative to some aspects, the delay layer includes a potassium binder disposed therein.

In a twenty-third aspect, in addition to one or more of the preceding or following aspects, or in the alternative to some aspects, the delay layer can be bioerodible.

In a twenty-fourth aspect, a chemical sensing device can be included having an optical transmission layer. The optical transmission layer can define a plurality of microneedles. The chemical sensing device can include a plurality of sensor elements and one or more optical emitters, wherein the one or more optical emitters can be configured to emit light into the plurality of sensor elements. The chemical sensing device can also include a plurality of optical detectors, wherein the plurality of optical detectors can be configured to receive light from the plurality of sensor elements.

In a twenty-fifth aspect, in addition to one or more of the preceding or following aspects, or in the alternative to some aspects, the optical transmission layer can define a plurality of sensing wells.

In a twenty-sixth aspect, in addition to one or more of the preceding or following aspects, or in the alternative to some aspects, wherein each of the plurality of sensing wells can be in fluid communication with at least one of the plurality of microneedles.

In a twenty-seventh aspect, in addition to one or more of the preceding or following aspects, or in the alternative to some aspects, at least some of the plurality of optical detectors can be disposed over a top surface of the optical transmission layer.

In a twenty-eighth aspect, in addition to one or more of the preceding or following aspects, or in the alternative to some aspects, at least some of the plurality of optical detectors can be disposed over a lateral side surface of the optical transmission layer.

In a twenty-ninth aspect, in addition to one or more of the preceding or following aspects, or in the alternative to some aspects, the plurality of sensor elements can be configured to detect at least one analyte, and the at least one analyte can include at least one selected from the group consisting of potassium ion, sodium ion, hydrogen ion (pH), and creatinine.

In a thirtieth aspect, in addition to one or more of the preceding or following aspects, or in the alternative to some aspects, the chemical sensing device can further include a pH sensor.

In a thirty-first aspect, in addition to one or more of the preceding or following aspects, or in the alternative to some aspects, the pH sensor can be integrated with or separate from the plurality of sensor elements.

In a thirty-second aspect, in addition to one or more of the preceding or following aspects, or in the alternative to some aspects, the chemical sensing device can further include a temperature sensor.

In a thirty-third aspect, in addition to one or more of the preceding or following aspects, or in the alternative to some aspects, the temperature sensor can be integrated with or separate from the plurality of sensor elements.

In a thirty-fourth aspect, in addition to one or more of the preceding or following aspects, or in the alternative to some aspects, the chemical sensing device can further include an adhesive patch, wherein the adhesive patch can be configured to hold the chemical sensing device onto the skin of a patient.

In a thirty-fifth aspect, in addition to one or more of the preceding or following aspects, or in the alternative to some aspects, the chemical sensing device can further include a control circuit, wherein the control circuit can be configured to control the one or more optical emitters and receive signals from the plurality of optical detectors.

In a thirty-sixth aspect, in addition to one or more of the preceding or following aspects, or in the alternative to some aspects, the sensor elements can include a chromoionophore sensing material.

In a thirty-seventh aspect, a method of measuring a concentration of an analyte with a wearable device can be included. The method can include taking a sample of a bodily fluid with a microneedle array, allowing the analyte from the sample to infiltrate a chemical sensor element, adjusting a pH of the chemical sensor element, and measuring a concentration of the analyte optically using the chemical sensor element.

In a thirty-eighth aspect, in addition to one or more of the preceding or following aspects, or in the alternative to some aspects, adjusting a pH of the chemical sensor element includes contacting the chemical sensor element with a pH adjusting solution.

In a thirty-ninth aspect, in addition to one or more of the preceding or following aspects, or in the alternative to some aspects, contacting the chemical sensor element with a pH adjusting solution includes opening a valve and/or actuating a pump to cause flow of the pH adjusting solution.

In a fortieth aspect, in addition to one or more of the preceding or following aspects, or in the alternative to some aspects, the analyte can include at least one selected from the group consisting of potassium ion, sodium ion, calcium ion, and a protein.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a patient with a chemical sensing device in accordance with various embodiments herein.

FIG. 2 is a schematic view of components of a chemical sensing device in accordance with various embodiments herein.

FIG. 3 is a schematic view of components of a chemical sensing device in accordance with various embodiments herein.

FIG. 4 is a schematic view of components of a chemical sensing device in accordance with various embodiments herein.

FIG. 5 is a schematic view of components of a chemical sensing device in accordance with various embodiments herein.

FIG. 6 is a schematic view of components of a chemical sensing device in accordance with various embodiments herein.

FIG. 7 is a flow chart of operations performed in accordance with various embodiments herein.

FIG. 8 is a block diagram of components of a chemical sensing device in accordance with various embodiments herein.

FIG. 9 is a schematic cross-sectional view of portions of a chemical sensing device in accordance with various embodiments herein.

FIG. 10 is a schematic cross-sectional view of portions of a chemical sensing device in accordance with various embodiments herein.

FIG. 11 is a schematic top view of a chemical sensing device in accordance with various embodiments herein.

FIG. 12 is a schematic cross-sectional view of portions of a chemical sensing device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Implantable analyte sensors can be used to gather data about physiological analytes while a patient is away from a medical care facility and without needing to draw blood or another fluid from the patient. However, maximum device volume, complexity and surface area are severely constrained in the context of implanted devices which limits both functionality and device longevity. Wearable sensors offer various benefits while avoiding many of the constraints of implanted devices. For example, wearable sensors offer the ability to gather data while the patient is away from a medical care facility while also avoiding the inconvenience of an implant procedure. Wearable sensors are also ideal for short-term use cases. However, the design of wearable analyte sensors remains challenging.

Embodiments of optical chemical sensor systems herein include wearable configurations that include microneedle arrays. Such wearable optical chemical sensor systems herein can provide accurate and rapid measurements of various analytes of medical interest.

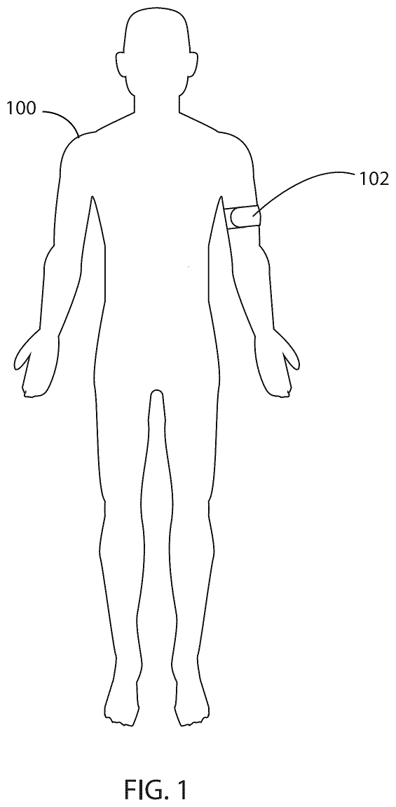

Referring now to FIG. 1, a schematic view of a patient 100 with a chemical sensing device 102 is shown in accordance with various embodiments herein. As described further below, in various embodiments the chemical sensing device 102 can include a set of microneedles and a sensor element. In some embodiments, the sensor element can include a first low-index (low refractive index) gel layer, a second low-index (low refractive index) gel layer, and a chromoionophore sensing film disposed there between. In some embodiments, refractive index values for the gel layers can be less than 1.8, 1.7, 1.6, 1.5, 1.4, 1.3, 1.2, or 1.1. In some embodiments, the refractive index value can fall within a range between any of the foregoing. In some embodiments, a delay layer can be disposed between the sensor element and the set of microneedles. An optical emitter can be configured to emit light into the sensor element and an optical detector can be configured to receive light from the sensor element. The received light can be processed by the chemical sensing device 102 to determine a concentration of an analyte of interest. In various embodiments, the chemical sensing device 102 can be configured to detect at least one analyte, such as at least one analyte selected from potassium ion, sodium ion, hydrogen ion (pH), and creatinine. However, other analytes can also be detected as described further below.

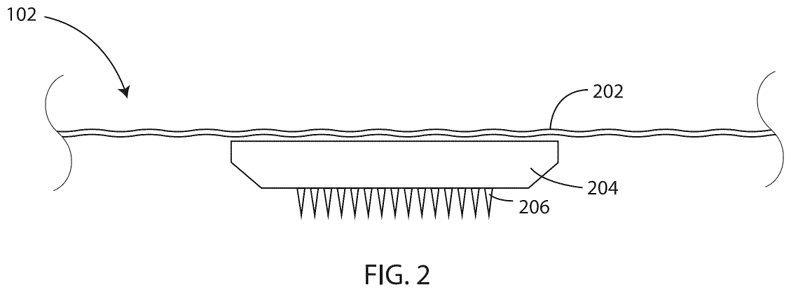

Referring now to FIG. 2, a schematic view of components of a chemical sensing device 102 is shown in accordance with various embodiments herein. In this embodiment, the chemical sensing device 102 includes an adhesive patch 202. In various embodiments, the adhesive patch 202 can be configured to hold the chemical sensing device 102 onto the skin of a patient 100. However, in some embodiments, the chemical sensing device 102 can include a strap or other mechanism to retain the chemical sensing device on the skin of the patient. The chemical sensing device 102 as shown includes a sensor housing 204 and an array of microneedles 206. Exemplary microneedles herein can range from 25 to 2500 μm in length, 20 to 250 μm in width, and 1 to 25 μm in tip diameter. Microneedles herein can be formed using various metals, silicon, various glasses, and/or various polymers.

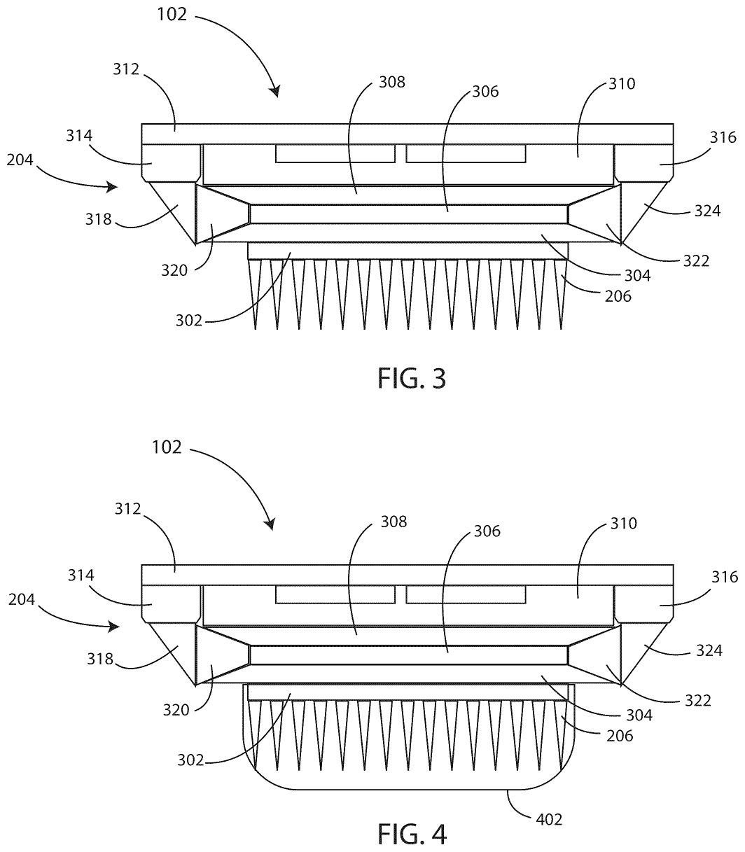

Referring now to FIG. 3, a schematic view of components of a chemical sensing device 102 is shown in accordance with various embodiments herein. As before, the chemical sensing device 102 includes a sensor housing 204 and a set of microneedles 206. In various embodiments, the microneedles 206 can penetrate at least part of the epidermis of the patient to allow for a flow of a fluid (such as interstitial fluid) containing an analyte of interest up through the microneedles 206 and into the sensor housing 204.

In this example, the chemical sensing device also includes a delay layer 302. The delay layer 302 is disposed between the microneedles 206 and a sensor element in the sensor housing 204. In various embodiments, the delay layer 302 can be effective to block or delay migration of a solute(s) or analytes from a set of microneedles 206 to a sensor element. In various embodiments, the delay layer 302 can be degradable (or biocrodible) and/or dissolvable and can begin to breakdown and/or dissolve after initial exposure to a fluid or component passing through the microneedles and reaching the delay layer 302. After the delay layer 302 breaks down or dissolves sufficiently, then one or more solutes or analytes from the set of microneedles 206 can pass on to the sensor element for detection. In some embodiments, the delay layer 302 can be formed of a hydrogel, degradable polymer (such as a polymer with bonds that break down through hydrolysis or another mechanism under conditions associated with contact with a fluid passing through the microneedles), dissolvable substance, or the like. In some embodiments, the delay layer 302 can include a potassium binder disposed therein.

The chemical sensing device also includes an optical emitter 314 and an optical detector 316. In various embodiments, the optical emitter 314 can be configured to emit light into a sensor element. In various embodiments, the optical detector 316 can be configured to receive light from a sensor element. Details of exemplary optical emitters and optical detectors are provided below.

The chemical sensing device 102 can include various components to guide and/or filter light. In various embodiments, at least one filter guide and at least one reflector are configured to be disposed optically between (e.g., along a light path between) at least one of an optical emitter or an optical detector and a sensor element. In the example of FIG. 3, the chemical sensing device includes a first reflector 318, a first filter guide 320, a second reflector 324, and a second filter guide 322.

The sensor element portion of the chemical sensing device 102 can include a first low-index (refractive index) gel layer 304, a chromoionophore containing sensing film 306, and a second low-index (refractive index) gel layer 308. Exemplary chemistries of the chromoionophore sensing film 306 are described in greater detail below. In various embodiments, the chromoionophore sensing film 306 can be disposed between the first low-index gel layer 304 and a second low-index gel layer 308. In some embodiments, low-index gel layers herein can have a refractive index of less than 1.6, 1.5, 1.4, 1.3, 1.2, 1.1 or less, or a refractive index falling within a range between any of the foregoing.

The chemical sensing device also includes a circuit board 312. In some embodiments, the chemical sensing device can also include an overfill circuit package 310 below the circuit board 312. In some embodiments, space can be included to allow for displacement space as fluid enters the chemical sensing device.

Referring now to FIG. 4, a schematic view of components of a chemical sensing device 102 is shown in accordance with various embodiments herein. FIG. 4 includes components shown in FIG. 3 including a sensor housing 204, a set of microneedles 206, delay layer 302, an optical emitter 314, an optical detector 316, a first reflector 318, a first filter guide 320, a second reflector 324, and a second filter guide 322. However, in FIG. 4, the chemical sensing device also includes a cover 402. The cover 402 can be used to protect the microneedles 206 until the time of use.

In some embodiments, the testing medium (fluid surrounding the chromoionophore sensing film) must be within a particular pH range in order for the sensing chemistry to work properly. For example, in some embodiments, the testing medium must be at a relatively acidic pH compared with normal physiological pH values. In some embodiments herein, a composition can be used to lower the pH of the testing medium. For example, an acidic composition can be used to lower the pH to a proper level for testing, such as a pH of 3 to 5, or about 4.

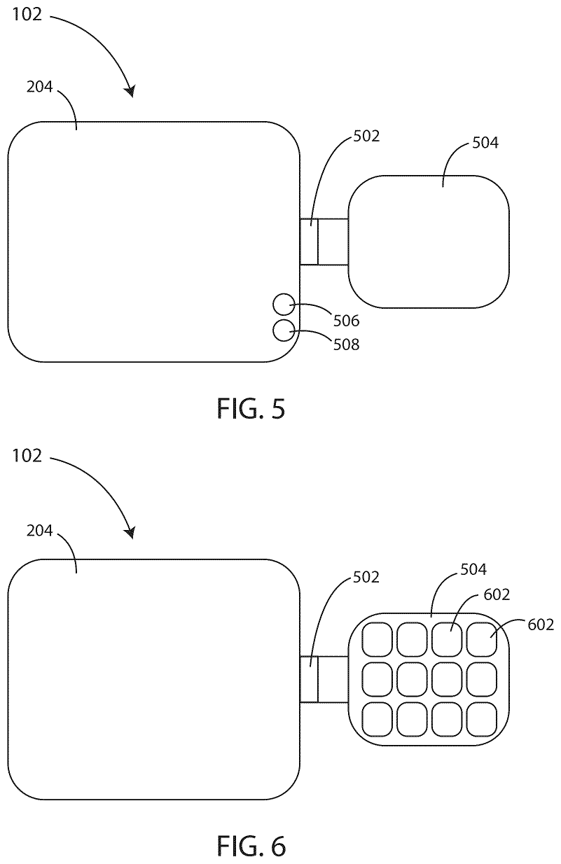

Referring now to FIG. 5, a schematic view of components of a chemical sensing device 102 is shown in accordance with various embodiments herein. The chemical sensing device includes a sensor housing 204, a flow control element 502, and a liquid reservoir 504.

In various embodiments, the flow control element 502 can be in fluid communication with the liquid reservoir 504 and can be configured to control a flow of a pH modifying composition from the liquid reservoir 504 to a sensor element (and/or to other components such as a delay hydrogel layer. Thus, the liquid reservoir 504 can be in selective fluid communication with at least one of a sensor element and a delay hydrogel layer.

In various embodiments, the flow control element 502 can include an actuatable element, such as an actuatable valve. In various embodiments, the flow control element 502 can include a pump. In various embodiments, the flow control element 502 can include a mechanism to change a pressure within the liquid reservoir 504, such as through mechanical and/or thermal means. In various embodiments, the flow control element 502 can include a one-way pressure valve, a heating pad, a magnetic valve, or the like.

In various embodiments, the pH modifying composition can include a carboxylic acid. In various embodiments, the carboxylic acid can be a biocompatible carboxylic acid. In various embodiments, the carboxylic acid can include citric acid. In various embodiments, the pH modifying composition can include a biocompatible buffer system.

In some embodiments, chemical sensing devices herein can include various other sensors that can be helpful in processing signals in order to generate accurate measurements of analytes. By way of example, as pH can influence signals generated herein, the chemical sensing device can include a pH sensor 506, which can be disposed inside the sensor housing 204 and configured to be in fluid communication with fluids therein. pH sensors can include combination pH sensors, differential pH sensors, ISFET electrode based sensors, and the like. The pH sensor 506 can be in signal communication with the control circuitry of the chemical sensing device. Temperature can also influence signals generated herein. As such, in some embodiments, the chemical sensing device can include a temperature sensor 508, which can be disposed in, on, adjacent to, or remotely from the sensor housing 204. Temperature sensors can include thermistors, thermocouples, resistive temperature detectors (RTDs), semiconductor based ICs, and the like. The temperature sensor 508 can be in signal communication with the control circuitry of the chemical sensing device.

In some embodiments, the liquid reservoir can include discrete separately controllable liquid portions. Referring now to FIG. 6, a schematic view of components of a chemical sensing device 102 is shown in accordance with various embodiments herein. As before, the chemical sensing device includes a sensor housing 204, a flow control element 502, and a liquid reservoir 504. In this embodiment, the liquid reservoir 504 includes a discrete separately controllable liquid portions 602.

### Methods

Many different methods are contemplated herein, including, but not limited to, methods of making, methods of using, and the like. In various embodiments, methods of measuring the concentration of an analyte of interest are included herein. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented as instructions stored on a non-transitory, computer-readable medium that, when executed by one or more processors, cause a system to execute the operations and/or steps.

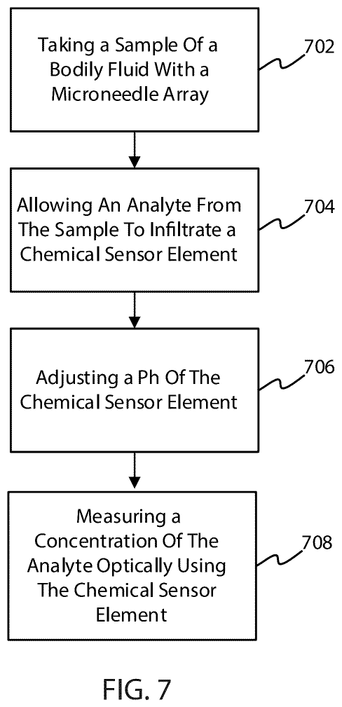

Referring now to FIG. 7, a flow chart of some operations performed is shown in accordance with a method of measuring a concentration of an analyte with a wearable device. In some embodiments the method can include an operation of taking a sample of a bodily fluid with a microneedle array 702. The method can also include an operation of allowing an analyte from the sample to infiltrate a chemical sensor element 704. The method can also include an operation of adjusting a pH of the chemical sensor element 706. The method of measuring a concentration of an analyte with a wearable device can also include an operation of measuring a concentration of the analyte optically using the chemical sensor element 708.

In an embodiment of the method, adjusting a pH of the chemical sensor element comprises contacting the chemical sensor element with a pH adjusting solution. In an embodiment of the method, an operation can include opening a valve and/or actuating a pump to cause flow of the pH adjusting solution.

### Further Device Components

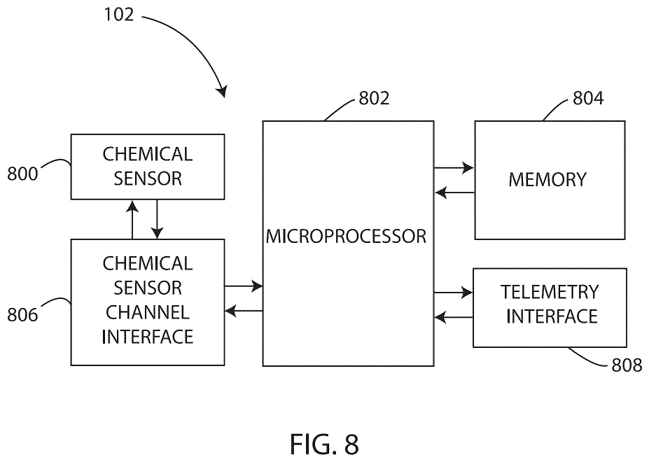

Referring now to FIG. 8, a schematic diagram of components of a chemical sensing device 102 in accordance with various embodiments herein. It will be appreciated that some embodiments can include additional elements beyond those shown in FIG. 8. In addition, some embodiments may lack some elements shown in FIG. 8. The chemical sensing device 102 can gather information through one or more sensing channels. A microprocessor 802 can communicate with a memory 804 via a bidirectional data bus. The memory 804 can include read only memory (ROM) or random access memory (RAM) for program storage and RAM for data storage, or any combination thereof. The chemical sensing device 102 can also include one or more analyte sensors 800 and one or more analyte sensor channel interfaces 806 which can communicate with a port of microprocessor 802. The analyte sensor channel interface 806 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like. A telemetry interface 808 is also provided for communicating with external devices such as a programmer, a home-based unit, and/or a mobile unit (e.g., a cellular phone, portable computer, etc.), implanted devices such as a pacemaker, cardioverter-defibrillator, loop recorder, and the like. The telemetry interface can include components (transmitters, receivers, transceivers, antennas, etc.) for wireless communication using various frequencies and protocols including WIFI, BLUETOOTH, an ISM band radio link, or the like. In various embodiments, the system can include a power supply circuit which can be configured to utilize a battery (rechargeable or non-rechargeable) and/or can be configured to receive a power input (wired or wireless power transmission). In some embodiments, electronic components of the chemical sensing device (such as the optical emitter, optical receiver, telemetry components, power supply circuit, etc.) can be part of an electronic package that can be reusable and other components of the chemical sensing device (such as microneedles) can be replaced for subsequent use.

### Further Embodiments

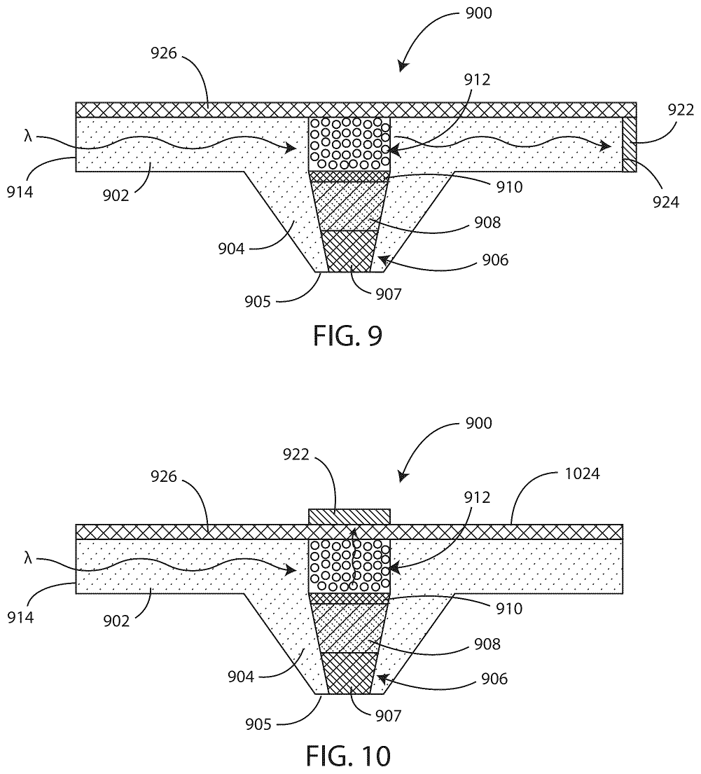

Chemical sensing devices herein can take on many different physical configurations. In some embodiments, a needle or needle-like structure can be integrated with a structure through which light passes. While not intending to be bound by theory, integration of a needle or needle-like structure with a structure through which light passes can provide an efficient and manufacturable structure where many needles can be formed interfacing with an array of sensor elements. As an example, referring now to FIG. 9 a schematic view is shown of portions of a chemical sensing device in accordance with various embodiments herein where needle structures are formed with a material through with light passes for purposes of chemical sensing herein. The chemical sensing device 900 can include an optical transmission layer, such as in the form of wafer or substrate 902 formed of a material through which light at wavelengths of interest herein passes (such as a polymer, a glass, a ceramic or the like). In some embodiments, portions of the wafer or substrate 902 can exhibit total internal reflection, such as where light is being conveyed to or away from an optoactive well or sensing well. The wafer or substrate 902 can also form a needle structure 904 with a tip 905 and an aperture in the tip 905 leading to a hollow internal portion 906. As such, the optical transmission layer can define one or more microneedles. In some embodiments, the wafer or substrate 902 can be substantially planar other than the microneedles. Various components can be disposed within the hollow internal portion 906 such as one or more of a delay layer 907, a filter plug 908, and/or a membrane barrier 910. The filter plug 908 can function to allow analytes of interest to pass through while keeping other components out. Further, a sensing well (or optoactive well) 912 can be disposed within the wafer or substrate 902. The sensing well can be in fluid communication with the needle structure 904 such that fluids and/or analytes entering the aperture in the tip 905 thereof can migrate to the sensing well 912. Sensor element components can be disposed in the sensing well 912, such as sensor element components including a chromoionophore or another component of a sensor clement herein. The membrane barrier 910 can function to keep components of the sensor element (such as when the sensor element includes particles) within the sensing well 912. A top barrier layer 926 can seal the top of the sensing well 912 and/or can serve as an optical shroud to prevent light from entering from the top side.

It will be appreciated that in some embodiments the delay layer 907 may not be confined to being within the hollow internal portion 906 of the needle structure 904. For example, the delay layer 907 could also be disposed covering an outer surface of the needle structure 904 apart from the aperture in the tip 905. In some embodiments, the delay layer 907 covers a portion of an outer surface of the needle structure 904 including an aperture in the tip 905, but is not within the hollow internal portion of the needle structure 904.

In operation, light can enter the wafer or substrate 902 from a first side 914 (or first lateral side) then pass through the wafer or substrate 902 to the sensing well 912. After interfacing with sensor element components in the sensing well 912 then the light can continue on to an optical detector 922 that, in this embodiment, is disposed on a second side 924 (or second lateral side) of the wafer or substrate 902. In some embodiments, the second side 924 is opposite from the first side 914.

It will be appreciated, however, that optical detectors can be placed in various positions with respect to the wafer or substrate 902. For example, in some embodiments optical detectors can be placed on a top side of the wafer or substrate 902. Referring now to FIG. 10 portions of a chemical sensing device are shown in accordance with various embodiments herein. As with the embodiment shown in FIG. 9, the chemical sensing device 900 includes wafer or substrate 902, needle structure 904, tip 905, hollow internal portion 906, delay layer 907, filter plug 908, membrane barrier 910, sensing well 912, and first side 914. However, in the embodiment shown in FIG. 10, the optical detector 922 is disposed over a top surface 1024 of the wafer or substrate 902 and top barrier layer 926.

The water or substrate 902 can be formed having dimensions sufficient such that there can be an array of needles, wells, sensor elements and/or optical detectors.

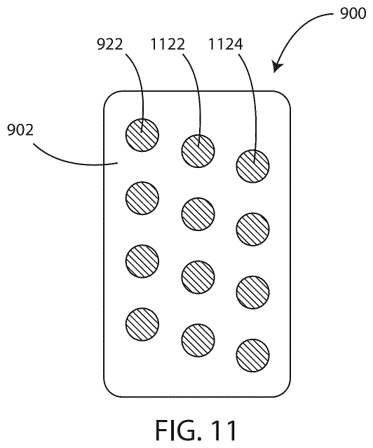

Referring now to FIG. 11, a top view is shown of portions of a chemical sensing device 900 herein. In this example, the chemical sensing device 900 includes wafer or substrate 902 and optical detectors 922, 1122, 1124 forming a detector array over the top of wafer or substrate 902. One or more (or an array) of optical emitters (not shown in this view) can be disposed on one or more lateral sides of the wafer or substrate 902.

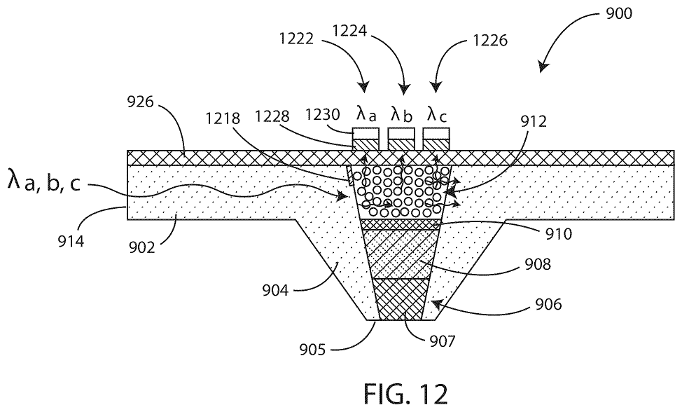

In some embodiments, optical detectors with sensitivities to different wavelengths of light can be used. Referring now to FIG. 12, portions of a chemical sensing device 900 are shown in accordance with various embodiments herein. As with the embodiment shown in FIG. 9, the chemical sensing device 900 includes wafer or substrate 902, needle structure 904, tip 905, hollow internal portion 906, delay layer 907, filter plug 908, membrane barrier 910, sensing well 912, and first side 914. However, in this embodiment, a first optical detector 1222 is included having a first optical filter 1228 and a first detector component 1230. A second optical detector 1224 is included also having an optical filter and a second detector component. The optical filter of the second optical detector 1224 can be different than the first optical filter 1228 in terms of wavelengths of light that can pass there through such that the second optical detector 1224 can be sensitive for different wavelengths of light than the first optical detector 1222. A third optical detector 1226 is included also having an optical filter and a second detector component. The optical filter of the third optical detector 1226 can be different than the first optical filter 1228 and the second optical filter in terms of wavelengths of light that can pass there through such that the third optical detector 1226 can be sensitive for different wavelengths of light than the first optical detector 1222 and the second optical detector 1224. In some embodiments, different optical emitters can be used emitting light at different frequencies and/or different frequency bands.

In some embodiments, one or more occlusion masks 1218 can be used in order to prevent light from entering sensing well 912 from certain directions such as to prevent ambient light from entering sensing well 912. In some embodiments, one or more light guides or light pipes can be disposed within the wafer or substrate 902. For example, in some embodiments, a light guide or light pipe can be used to convey light from a particular optical emitter to a particular sensing well 912 and/or from a particular sensing well 912 to a particular optical detector.

### Analyte Sensors

Analyte sensors herein can be of various types. In some embodiments, the physiological concentration of an analyte is sensed directly. In other embodiments, the physiological concentration of an analyte is sensed indirectly. By way of example, a metabolite of a particular analyte can be sensed instead of the particular analyte itself. In other embodiments, an analyte can be chemically converted into another form in order to make the process of detection easier. By way of example, an enzyme can be used to convert an analyte into another compound that is easier to detect. For example, the hydrolysis of creatinine into ammonia and N-methylhydantoin can be catalyzed by creatinine deiminase and the resulting ammonia can be detected by an analyte sensor.

Analyte sensors herein can include optical devices that utilize changes of optical phenomena or properties, which are the result of an interaction of the analyte with at least part of the sensor. Such optical properties can include: absorbance, caused by the absorptivity of the analyte itself or by a reaction with some suitable indicator; reflectance, using a bodily component, tissue, or fluid, or using an immobilized indicator; photoluminescence, based on the measurement of the intensity of light emitted by a chemical reaction in the receptor system; fluorescence, measured as the positive emission effect caused by irradiation or selective quenching of fluorescence; refractive index, measured as the result of a change in solution composition, in some cases including surface plasmon resonance effects; optothermal effects, based on a measurement of the thermal effect caused by light absorption; light scattering; or the like. In some embodiments, optical analyte sensors can include an optode.

Analyte sensors can also include electrochemical devices that transform the effect of the electrochemical interaction between an analyte and an electrode into a useful signal. Such sensors can include voltammetric sensors, including amperometric devices. Also included are sensors based on chemically inert electrodes, chemically active electrodes and modified electrodes. Also included are sensors with and without (galvanic sensors) a current source. Sensors can also include potentiometric sensors, in which the potential of the indicator electrode (ion-selective electrode, redox electrode, metal oxide electrode, or the like) is measured against a reference electrode. Sensors can include chemically sensitized field effect transistors (CHEMFET) in which the effect of the interaction between the analyte and the active coating is transformed into a change of the source-drain current. Sensors can include potentiometric solid electrolyte gas sensors.

In one example of the operation of an optical analyte sensor, analytes of interest from the in vivo environment can diffuse into the sensing film causing a detectable change in the optical properties of the sensing film. Light can be generated by an optical excitation device or emitter, such as an LED or similar device, and can pass through the optical window and into the analyte sensing element. Light can then either be preferentially reflected from or re-emitted by the sensing film proportionally to the sensed analyte and pass back through the optical window before being received by a light detection device or receiver, such as a charge-coupled device (CCD), a photodiode, a junction field effect transistor (JFET) type optical sensor, of complementary metal-oxide semiconductor (CMOS) type optical sensor. Various aspects of exemplary analyte sensors are described in greater detail in U.S. Pat. No. 7,809,441, the content of which is herein incorporated by reference in its entirety. In another example of the operation of an optical analyte sensor, the optical properties of a tissue or fluid in the body can be directly analyzed. By way of example, light can be generated by an optical excitation device that can be delivered to a component, tissue, or fluid in the body and a light detection device can be used to sense an optical property of the light that has interfaced with the component, tissue, or fluid.

In accordance with the embodiments herein, some sensing element(s) can include one or more ion-selective sensor components. Ion-selective components may either rely on surface phenomena or on concentration changes inside the bulk of a phase. Ion-selective sensors can include optical sensors, including both non-carrier optical sensors and carrier-based optical sensors, and ion-selective electrodes (ISEs). In some embodiments, the ion-selective sensor is fluorimetric, and can include a complexing moiety and a fluorescing moiety. Fluorimetric ion-selective sensors can exhibit differential fluorescent intensity based upon the complexing of an analyte to a complexing moiety. In some embodiments, the ion-selective sensor can be colorimetric, and can include a complexing moiety and a colorimetric moiety. Colorimetric ion-selective sensors can exhibit differential light absorbance based upon the complexing of an analyte to a complexing moiety.

In some embodiments, the ion-selective sensor comprises a non-carrier or carrier-based fluorescent or colorimetric ionophoric composition that comprises a complexing moiety for reversibly binding an ion to be analyzed, and a fluorescing or colorimetric moiety that changes its optical properties as the complexing agent binds or releases the ion. The complexing agents of the invention can optionally be appended with one or more organic substituents chosen to confer desired properties useful in formulating the ion sensing composition. By way of example, the substituents can be selected to stabilize the complexing agent with respect to leaching into the solution to be sensed, for example, by incorporating a hydrophobic or polymeric tail or by providing a means for covalent attachment of the complexing agent to a polymer support within the ion-selective sensor.

In some embodiments, the sensing element can include ion-selective sensor components such as an ionophore or a fluorionophore. Suitable ionophores for use with the embodiments herein can include, but not be limited to, sodium specific ionophores, potassium specific ionophores, calcium specific ionophores, magnesium specific ionophores, and lithium specific ionophores. Suitable fluorionophores for use with the embodiments herein can include, but not be limited to, lithium specific fluoroionophores, sodium specific fluoroionophores, and potassium specific fluoroionophores.

Exemplary ion-selective sensing components and methods for their use are disclosed in commonly assigned U.S. Pat. No. 7,809,441, the contents of which is herein incorporated by reference in its entirety.

### Colorimetric and Photoluminescent Chemistries

Colorimetric sensing components herein can be specific for a particular chemical analyte. Colorimetric sensing components can include an element that changes color based on binding with or otherwise complexing with a specific chemical analyte. In some embodiments, a colorimetric response element can include a complexing moiety and a colorimetric moiety. Those moieties can be a part of a single chemical compound (as an example a non-carrier based system) or they can be separated on two or more different chemical compounds (as an example a carrier based system). The colorimetric moiety can exhibit differential light absorbance on binding of the complexing moiety to an analyte.

Photoluminescent sensing components herein can be specific for a particular chemical analyte. Photoluminescent components herein can include an element that absorbs and emits light through a photoluminescent process, wherein the intensity and/or wavelength of the emission is impacted based on binding with or otherwise complexing with a specific chemical analyte. In some embodiments, a photoluminescent response element can include a complexing moiety and a fluorescing moiety. Those moieties can be a part of a single chemical compound (as an example a non-carrier based system) or they can be separated on two or more different chemical compounds (as an example a carrier based system). In some embodiments, the fluorescing moiety can exhibit different fluorescent intensity and/or emission wavelength based upon binding of the complexing moiety to an analyte.

Some chemistries may not require a separate compound to both complex an analyte of interest and produce an optical response. By way of example, in some embodiments, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy) thiophenyl]coumarin, a potassium ion selective fluoroionophore, can be used (and in some cases covalently attached to polymeric matrix or membrane) to produce a fluorescence-based K⁺ non-carrier response element.

An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

Suitable fluoroionophores include the coumarocryptands taught in U.S. Pat. No. 5,958,782, the disclosure of which is herein incorporated by reference. Such fluorescent ionophoric compounds can be excited with GaN blue light emitting diodes (LEDs) emitting light at or about 400 nm. These fluorescent ionophoric compounds have ion concentration dependent emission that can be detected in the wavelength range of about 450 nm to about 470 nm.

Some chemistries can rely upon a separate complexing entity (e.g., a separate chemical compound). As an example, carrier based response elements can include a compound, in some cases referred to as an ionophore, that complexes with and serves to carry the analyte of interest. Both non-carrier based components and carrier-based response components can include complexing moieties. Suitable complexing moieties can include include cryptands, crown ethers, bis-crown ethers, calixarenes, noncyclic amides, and hemispherand moieties as well as ion selective antibiotics such as monensin, valinomycin and nigericin derivatives.

Those of skill in the art can recognize which cryptand and crown ether moieties are useful in complexing particular cations, although reference can be made to, for example, Lehn and Sauvage, “[2]-Cryptates: Stability and Selectivity of Alkali and Alkaline-Earth Macrocyclic Complexes,” J. Am. Chem. Soc, 97, 6700-07 (1975), for further information on this topic. Those skilled in the art can recognize which bis-crown ether, calixarene, noncyclic amides, hemispherand, and antibiotic moieties are useful in complexing particular cations, although reference can be made to, for example, Buhlmann et al., “Carrier-Based Ion-Selective Electrodes and Bulk Optodes. 2. Ionophores for Potentiometric and Optical Sensors,” Chem. Rev. 98, 1593-1687 (1998), for further information on this topic.

By way of example cryptands can include a structure referred to as a cryptand cage. For cryptand cages, the size of the cage is defined by the oxygen and nitrogen atoms and the size makes cryptand cages quite selective for cations with a similar diameter. For example, a [2.2.2] cryptand cage is quite selective for cations such as K⁺, Pb⁺², Sr⁺², and Ba⁺². A [2.2.1] cryptand cage is quite selective for cations such as Na⁺ and Ca⁺². Finally, a [2.1.1] cryptand cage is quite selective for cations such as Li⁺ and Mg⁺². The size selectivity of cryptand cages can aid in the sensitivity of chemical sensing. When these cryptand cages are incorporated into physiologic sensing systems heavier metals such as Pb⁺² and Ba⁺² are unlikely to be present in concentrations which interfere with the analysis of ions of broader physiological interest such as Na⁺ and K⁺.

Further aspects of colorimetric and photoluminescent sensors and components thereof are described in U.S. Pat. Nos. 7,809,441 and 8,126,554, the content of which is herein incorporated by reference.

### Optical Emitters and Detectors

In some embodiments, the optical emitter can include solid state light sources such as GaAs, GaAlAs, GaAlAsP, GaAlP, GaAsP, GaP, GaN, InGaAlP, InGaN, ZnSe, or SiC light emitting diodes or laser diodes that excite the sensing element(s) at or near the wavelength of maximum absorption for a time sufficient to emit a return signal. However, it will be understood that in some embodiments the wavelength of maximum absorption/reflection varies as a function of concentration in the colorimetric sensor.

In some embodiments, the optical emitter can include other light emitting components including incandescent components. In some embodiments, the optical emitter can include a waveguide. The optical excitation assemblies can also include one or more bandpass filters, high pass filter, low pass filter, antireflection elements, and/or focusing optics.

In some embodiments, the optical emitter can include a plurality of LEDs with bandpass filters, each of the LED-filter combinations emitting at a different center frequency. According to various embodiments, the LEDs can operate at different center-frequencies, sequentially turning on and off during a measurement, illuminating the sensing element(s). As multiple different center-frequency measurements are made sequentially, a single unfiltered detector can be used in some embodiments. However, in some embodiments, a polychromatic source can be used with multiple detectors that are each bandpass filtered to a particular center frequency.

In some embodiments, sensing components herein can include one or more types of indicator beads having embedded therein various types of ion-selective sensors. Physiological analytes of interest can diffuse into and out of the sensing element(s) and bind with an ion-selective sensor to result in a fluorimetric or colorimetric response. Reference analytes can similarly diffuse into and out of the sensing element(s) and serve as a control sample. Exemplary ion-selective sensors are described more fully below.

The optical detector (or detection assembly) can be configured to receive light from the sensing element(s). In an embodiment, the optical detection assembly can include a component to receive light. By way of example, in some embodiments, the optical detection assembly can include a charge-coupled device (CCD). In other embodiments, the optical detection assembly can include a photodiode, a junction field effect transistor (JFET) type optical sensor, or a complementary metal-oxide semiconductor (CMOS) type optical sensor. In some embodiments, the optical detection assembly can include an array of optical sensing components. In some embodiments, the optical detection assembly can include a waveguide. In some embodiments, the optical detection assembly can also include one or more bandpass filters and/or focusing optics. In some embodiments, the optical detection assembly can include one or more photodiode detectors, each with an optical bandpass filter tuned to a specific wavelength range.

The optical emitters and optical detectors embodied herein, can be integrated using bifurcated fiber-optics that direct excitation light from a light source to one or more sensing element(s), or simultaneously to sensing element(s) and a reference channel. Return fibers can direct emission signals from the sensing element(s) and the reference channels to one or more optical detectors for analysis by a processor, such as a microprocessor. In some embodiments, the optical emitter and optical detectors are integrated using a beam-splitter assembly and focusing optical lenses that direct excitation light from a light source to the sensing component and direct emitted or reflected light from the sensing component to an optical detector for analysis by a processor.

### Ion-Permeable Polymeric Matrix Materials

Various components of devices herein such as one or more of the delay gel layer, the low-index layers of the chemical sensor, and the like can be formed of an ion-permeable polymeric matrix material in some embodiments. Suitable polymers for use as the ion-permeable polymeric matrix material can include, but not be limited to polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In some embodiments, hydrogels herein can be prepared from polymerization of various monomers or macromers including one or more of 2-hydroxyethyl methacrylate (HEMA), 2-hydroxypropyl methacrylate (HPMA), acrylamide, acrylic acid, N-isopropylacrylamide (NIPAm), methoxyl polyethylene glycol monoacrylate (PEGMA), and the like. In some embodiments, polymers can include, but are not limited to polyhydroxyethyl methacrylate (polyHEMA), cellulose, polyvinyl alcohol, dextran, polyacrylamides, polyhydroxyalkyl acrylates, polyvinyl pyrrolidones, and mixtures and copolymers thereof. In some embodiments, suitable polymers for use with the ion-permeable polymeric matrix described herein include those that are transparent.

### Physiological Analytes

Examples of physiological analytes that can be measured in accordance with embodiments herein can include, but are not limited to, electrolytes, hormones, proteins, sugars, metabolites, and the like.

Sensors herein can be directed at a specific analyte or a plurality of different analytes. In an embodiment, the analyte sensed is one or more analytes relevant to cardiac health. In an embodiment, the analyte sensed is one or more analytes indicative of renal health. The analyte sensed can be ionic or non-ionic. The analyte sensed can be a cation or an anion. Specific examples of analytes that can be sensed include acetic acid (acetate), aconitic acid (aconitate), ammonium, blood urea nitrogen (BUN), B-type natriuretic peptide (BNP), bromate, calcium, carbon dioxide, cardiac specific troponin, chloride, choline, citric acid (citrate), cortisol, copper, creatinine, creatinine kinase, fluoride, formic acid (formate), glucose, hydronium ion, isocitrate, lactic acid (lactate), lithium, magnesium, maleic acid (maleate), malonic acid (malonate), myoglobin, nitrate, nitric-oxide, oxalic acid (oxalate), oxygen, phosphate, phthalate, potassium, pyruvic acid (pyruvate), selenite, sodium, sulfate, urea, uric acid, and zinc. Inorganic cations sensed by this method include but not limited to hydronium ion, lithium ion, sodium ion, potassium ion, magnesium ion, calcium ion, silver ion, zinc ion, mercury ion, lead ion and ammonium ion. Inorganic anions sensed by this method include but not limited to carbonate anion, nitrate anion, sulfite anion, chloride anion and iodide anion. Organic cations sensed by this method include but are not limited to norephedrine, ephedrine, amphetamine, procaine, prilocaine, lidocaine, bupivacaine, lignocaine, creatinine and protamine. Organic anions sensed by this method include but not limited to salicylate, phthalate, maleate, and heparin. Neutral analytes sensed by this method include but not limited to ammonia, ethanol, and organic amines. In an embodiment, ions that can be sensed include potassium, sodium, chloride, calcium, and hydronium (pH). In a particular embodiment, concentrations of both sodium and potassium are measured. In another embodiment, concentrations of both magnesium and potassium are measured.

In some embodiments, the analytes can specifically include one or more of sodium ion, magnesium ion, chloride ion, calcium ion, carbonate ion, phosphate ion, sulfate ion, insulin, aldosterone, troponin, glucose, creatinine, and BNP.

In some embodiments, the analytes can specifically include one or more of partial pressure of oxygen (PaO₂), partial pressure of carbon dioxide (PaCO₂) and oxygen saturation (O₂Sat).

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A chemical sensing device comprising:
an optical transmission layer, the optical transmission layer defining a plurality of microneedles;
a plurality of sensor elements in fluid communication with the microneedles;
one or more optical emitters, wherein the one or more optical emitters are configured to emit light into the plurality of sensor elements; and
a plurality of optical detectors, wherein the plurality of optical detectors are configured to receive light from the plurality of sensor elements.

2. The chemical sensing device of claim 1, the optical transmission layer defining a plurality of sensing wells.

3. The chemical sensing device of claim 2, wherein each of the plurality of sensing wells is in fluid communication with at least one of the plurality of microneedles.

4. The chemical sensing device of claim 3, wherein the sensing wells and microneedles are configured such that fluids and/or analytes entering the microneedle migrate to the sensing well.

5. The chemical sensing device of claim 2, wherein the sensor elements are disposed within the sensing wells.

6. The chemical sensing device of claim 2, further comprising a top barrier layer, wherein the top barrier layer is configured to seal a top of the sensing wells.

7. The chemical sensing device of claim 2, further comprising a top barrier layer, wherein the top barrier layer is configured as an optical shroud to prevent light from entering from a top side of the sensing wells.

8. The chemical sensing device of claim 1, wherein at least some of the plurality of optical detectors are disposed over a top surface of the optical transmission layer.

9. The chemical sensing device of claim 1, wherein at least some of the plurality of optical detectors are disposed over a lateral side surface of the optical transmission layer.

10. The chemical sensing device of claim 1, wherein at least portions of the optical transmission layer exhibit total internal reflection.

11. The chemical sensing device of claim 1, wherein the microneedles are formed of a material including one or more of a polymer, a glass, and a ceramic.

12. The chemical sensing device of claim 1, the microneedles comprising a tip, an aperture in the tip, and a hollow internal portion, wherein the aperture leads to the hollow internal portion.

13. The chemical sensing device of claim 12, further comprising a delay layer, wherein the delay layer is disposed within the hollow internal portion.

14. The chemical sensing device of claim 12, further comprising a filter plug, wherein the filter plug is disposed within the hollow internal portion.

15. The chemical sensing device of claim 12, further comprising a membrane barrier, wherein the membrane barrier is disposed within the hollow internal portion.

16. The chemical sensing device of claim 1, the sensor elements including a chromoionophore.

17. The chemical sensing device of claim 1, the sensor elements configured to sense at least one of electrolytes, hormones, proteins, sugars, metabolites.

18. The chemical sensing device of claim 1, the sensor elements configured to sense potassium.

19. The chemical sensing device of claim 1, wherein the chemical sensing device is a wearable device.

20. The chemical sensing device of claim 1, further comprising an adhesive patch, wherein the adhesive patch is configured to hold the chemical sensing device onto the skin of a patient.
